(19) United States
(12) Patent Application Publication
(10) Pub. No.: US 2006/0147448 A1
(43) Pub. Date: Jul. 6, 2006

(54) Treatment of immunological renal disorders by lymphotoxin pathway inhibitors

(76) Inventors: Jennifer Gommerman, Toronto (CA); Jeffrey Browning, Brookline, MA (US)

Correspondence Address: Biogen Idec, 14 Cambridge Center, Cambridge, MA, 02142, US

(21) Appl. No.: 10/533,153

(22) PCT Filed: Oct. 31, 2003
(86) PCT No.: PCT/US03/34813

§ 371 Date: Feb. 13, 2006

Related U.S. Application Data
(60) Provisional application No. 60/422,588, filed on Oct. 31, 2002.

(51) Int. Cl. A61K 39/395

(52) U.S. Cl. 424/143.1; 424/145.1

(57) ABSTRACT

The disclosure relates to treatment of immunological disorders, including but not limited to renal disorders associated with immunoglobulin deposits. The disclosure further relates to the lymphotoxin pathway. Compositions comprising lymphotoxin pathway inhibitors are described.

### RELATED APPLICATIONS

This application is related to U.S. Provisional Application No. 60/422,588, filed Oct. 31, 2002. The entire contents of which are hereby incorporated herein by reference.

### TECHNICAL FIELD

The technical field of the invention relates to treatment of immunological disorders, including but not limited to immunological renal disorders associated with immunoglobulin deposits. The field further relates to inhibition of the lymphotoxin pathway.

### BACKGROUND

Autoimmune diseases result from an abnormal immune response to self antigens. Generation of high affinity, somatically hypermutated auto-antibodies is one of the hallmarks of autoimmune conditions. Most autoimmune disorders involve renal manifestations associated with deposits of auto-reactive immunoglobulins in the kidneys. Some of these disorders ultimately result in renal failure. Glomerulonephritis associated with immunoglobulin deposits is particularly pronounced in chronic hepatitis, systemic lupus erythematosus (SLE), Henoch-Schonlein purpura (HSP), and IgA nephropathy also known as Berger's disease. A common component of these diseases is the presence of macroscopic or microscopic hematuria and mesangial IgA deposits, accompanied by glomerular damage. IgA immune complex deposits cause the most common form of human glomerulonephritis associated with a high incidence of renal failure (Berger (1969) Transplant. Proc. 1:939). SLE affects more than two million people in the United States alone. IgA nephropathy is found throughout the world but is most prevalent in Japan, Australia, Southeast Asia, and Southern Europe. In the U.S., the incidence is approximately 4% of all renal biopsies but may be as high as 45% of biopsies in Japan. Etiopathology of these diseases is poorly understood. There are currently no effective treatments for these diseases.

One approach to this problem is to test pathway inhibitors in a variety of animal models that employ different disease driving mechanisms. Progress in understanding and treating these diseases has been constrained by the unavailability of animal models that mimic all aspects of disease states.

### SUMMARY OF THE INVENTION

The invention provides methods for treating immunological disorders, including pathologies associated with immunoglobulin (Ig) deposits in the kidneys. The invention is based in part on the discovery that inhibition of lymphotoxin (LT) pathway with a soluble form of the LT-P receptor (LTBR) leads to amelioration of lupus-like disease in BAFF-transgenic mice. The invention is further based in part on the discovery that renal dysfunction in BAFF-transgenic mice is associated with accumulation of IgA and IgG immune complexes in the kidneys.

Thus, one aspect of the invention provides methods for treating immunological disorders, including diseases caused by dysregulated production of immunoglobulins by B cells, including dysregulated production of IgA or IgG. In some embodiments, compositions comprising inhibitors of the LT pathway are used to prevent or to treat pathologies associated with renal dysfunction accompanied by immunoglobulin deposits, including, but not limited to glomerulonephritis. The invention also provides assays for identifying and/or testing efficacy of a therapeutic compound in a nonhuman animal for treatment of IgA nephropathy and related pathologies. In some embodiments, the assay comprises administering a compound being tested to a BAFF-transgenic animal and determining the level of IgA deposits in BAFF-transgenic kidneys.

In certain embodiments, the inhibitors of the LT pathway used in the methods of the invention comprise LTBR derivatives such as soluble forms of LTBR, e.g., LTBR immunoglobulin fusion, antibodies against LTBR, or antibodies against the LTBR ligand, LT. In one embodiment of the invention, the inhibitor of the LT pathway comprises a soluble LTBR fused to one or more heterologous protein domains, including, but not limited to, a human immunoglobulin Fc domain. In another embodiment, the inhibitor comprises a soluble LTBR comprising a functional sequence of amino acids selected from the amino acids of SEQ ID NO: 1. In still another embodiment, the soluble LTBR comprises a ligand binding domain that can selectively bind to lymphotoxin (LT) ligand comprising at least one LT beta subunit. In still another embodiment, the soluble LTBR comprises an extracellular domain of LT-beta-R.

It is to be understood that both the foregoing general description and the following detailed description are exemplary and explanatory only and are not restrictive of the invention as claimed.

### BRIEF DESCRIPTION OF THE FIGURES

FIG. 1 demonstrates that treatment of diseased BAFF-transgenic (BAFF Tg) mice with LTBR-Ig results in amelioration of kidney function as indicated by the proteinurea (PU) score. Six-month old BAFF Tg mice and non-transgenic (Tg neg.) littermate controls received intraperitoneal (i.p.) injections of 100 μg of LTBR-Ig or 100 μg of human IgG (huIgG) once a week for 5 weeks. FIG. 1A shows absolute changes in the PU score at the completion of treatment. FIG. 1B shows relative changes in the PU score.

FIG. 2 demonstrates that treatment of diseased BAFF-Tg mice with LTBR-Ig results in amelioration of glomerular pathology as indicated by periodic acid-Schiff (PAS) histochemical staining of the kidneys. Six-month old BAFF Tg mice and Tg neg. littermate controls received i.p. injections of 100 μg of LTBR-Ig or 100 μg of huIgG once a week for 5 weeks. Kidneys were harvested for histologic analysis. Representative histochemical samples are shown in FIGS. 2A-2F. Glomerular pathology scores are shown in FIG. 2G.

FIG. 3 demonstrates the effect of LTBR-Ig treatment on auto-antibody titers. Six-month old BAFF Tg mice and Tg neg. littermate controls received i.p. injections of 100 μg of LTBR-Ig or 100 μg of huIgG once a week for 5 weeks. Serum Ig levels were measured and kidneys were harvested for histologic analysis. FIGS. 3A illustrates that BAFF-Tg mice exhibit elevated levels of serum IgG2a, IgA, and IgM as compared to wild-type littermate controls. FIGS. 3B-3C illustrates that treatment with LTBR-Ig decreases auto-antibody titers in BAFF Tg mice.

FIG. 4 illustrates immune complex deposits in the kidneys of BAFF-Tg mice. Six-month old BAFF Tg mice and Tg neg. littermate controls received i.p. injections of 100 μg of LTBR-Ig or 100 μg of huIgG once a week for 5 weeks. Kidneys were harvested for histologic analysis. Representative histochemical samples are shown in FIG. 4C. The appearance of IgA (FIG. 4A) and IgG (FIG. 4B) immune complexes in glomeruli of BAFF-Tg and Tg neg. mice was scored.

FIG. 5 illustrates that LTBR-Ig treatment reduces marginal zone (MZ) B cells. Six-month old BAFF Tg mice and Tg neg. littermate controls received i.p. injections of 100 μg of LTBR-Ig or 100 μg of huIgG once a week for 5 weeks. Kidneys were harvested for histologic analysis. Spleen weights for BAFF Tg and Tg neg. mice are shown in FIG. 5A; spleen cell counts, numbers of mature CD23+ follicular B cells, and MZ B cells numbers are shown FIGS. 5B, 5C, and 5D, respectively. Representative histochemical samples are shown in FIG. 5E.

FIG. 6 illustrates that LTBR-Ig treatment reduces the number of PNA-positive GCs in BAFF Tg spleens. Six-month old BAFF Tg mice and Tg neg. littermate controls received i.p. injections of 100 μg of LTBR-Ig or 100 μg of huIgG once a week for 5 weeks. Kidneys were harvested for histologic analysis. Representative histochemical samples are shown in FIG. 6A. The percentage of PNA-positive follicles as a number of total with LTBR-Ig or huIgG treatment is shown in FIG. 6B.

FIG. 7 illustrates that the partial reduction in PNA-positive GCs is related to disease reduction observed in BAFF Tg mice. Six-month old BAFF Tg mice and Tg neg. littermate controls received i.p. injections of 100 μg of anti-CD40 ligand antibody (MR1) or 100 μg of huIgG once a week for 5 weeks. Kidneys were harvested for histologic analysis. Spleen weights for BAFF Tg and Tg neg. mice are shown in FIG. 7A; numbers of follicular B cells, numbers of MZ B cells, and percentage of PNA-positive GCs in the spleens are shown FIGS. 7B, 7C, and 7D, respectively. Representative histochemical samples are shown in FIG. 7E. Kidney function was assessed by proteinuria as illustrated in FIG. 7F.

### DETAILED DESCRIPTION OF THE INVENTION

Definitions

The term “antibody,” as used herein, refers to an immunoglobulin or a part thereof, and encompasses any polypeptide comprising an antigen-binding site regardless of the source, method of production, and other characteristics. The term includes but is not limited to polyclonal, monoclonal, monospecific, polyspecific, non-specific, humanized, single-chain, chimeric, synthetic, recombinant, hybrid, mutated, and CDR-grafted antibodies. The term “antigen-binding domain” refers to the part of an antibody molecule that comprises the area specifically binding to or complementary to a part or all of an antigen. Where an antigen is large, an antibody may only bind to a particular part of the antigen. The “epitope,” or “antigenic determinant” is a portion of an antigen molecule that is responsible for specific interactions with the antigen-binding domain of an antibody. An antigen-binding domain may be provided by one or more antibody variable domains (e.g., a so-called Fd antibody fragment consisting of a V_{H} domain). An antigen-binding domain comprises an antibody light chain variable region (V_{L}) and an antibody heavy chain variable region (V_{H}). The term “LTBR antibody,” or “antibody against LTBR,” refers to any antibody that specifically binds to at least one epitope of LTBR. The terms “LT antibody,” “antibody against LT,” or “antibody against the LTBR ligand, LT” refer to any antibody that specifically binds to at least one epitope of LT.

The term “therapeutic compound,” or “therapeutic,” as used herein, means any compound capable of “inhibiting” the LT pathway, either by inhibiting LTBR or LT expression at the transcriptional, translational, or post-translational levels or by inhibiting the biological activity of LTBR or its functional ligand LT.

The terms “inhibitor,” “inhibit,” “neutralize,” “antagonize,” and their cognates refer to the ability of a compound to act as an antagonist of a certain reaction or activity. The term “inhibit” refers to a decrease in the expression of LTBR or LT, or activity of LTBR or LT in the presence of a therapeutic compound, relative to the expression or the activity in the absence of the same compound. The decrease in the expression level or the activity is preferably at least about 10%, 20%, 30%, 40%, 50%, 60%, 70%, 80%, 90%, or higher. The expression level or the activity of LTBR or LT can be measured as described herein or by techniques known in the art (see, e.g., Rooney (2000) Methods Enzymol., 322:345-363) and U.S. Pat. No. 6,403,087).

The terms “treatment,” “therapeutic method,” and their cognates refer to both therapeutic treatment and prophylactic/preventative measures. Those in need of treatment may include individuals already having a particular medical disorder as well as those who may ultimately acquire the disorder.

The term “immunological renal disorder” refers to a disease or condition that involves dysregulated production of immunoglobulins resulting in kidney pathology. Such disorders include but are not limited to systemic lupus erythematosus, Sjogren's syndrome, and rheumatoid arthritis, insulin dependent diabetes mellitus (IDDM), chronic hepatitis, Henoch-Schonlein purpura (HSP), IgA nephropathy (Berger's disease) as well as any other disease, the clinical manifestations of which include the presence of macroscopic or microscopic hematuria and renal immunoglobulin deposits.

The term “effective dose,” or “effective amount,” refers to that amount of a compound that results in amelioration of symptoms in a patient or a desired biological outcome, e.g., inhibition of the LT pathway. The effective amount can be determined as described in the subsequent sections.

The terms “polynucleotide,” “oligonucleotide,” “nucleic acid,” and “DNA” are used interchangeably herein and refer to deoxyribonucleic acid (DNA), and, where appropriate, ribonucleic acid (RNA). The term should also be understood to include nucleotide analogs, and single or double stranded polynucleotides. Examples of polynucleotides include but are not limited to plasmid DNA or fragments thereof, viral DNA or RNA, anti-sense RNA, etc. The term “plasmid DNA” refers to double stranded DNA that is circular. “Antisense” nucleic acid, as used herein, refers to a nucleic acid capable of hybridizing to a portion of a coding and/or noncoding region of mRNA by virtue of some sequence complementarity, thereby interfering with translation from the mRNA.

As used herein the term “hybridization under defined conditions” is intended to describe conditions for hybridization and washes under which nucleotide sequences that are significantly identical or homologous to each other remain bound to each other. The conditions are such that sequences, which are at least 50, 100, 150, 300, or more nucleotides long and at least about 70%, more preferably at least about 80%, even more preferably at least about 85-90% identical, remain bound to each other. The percent identity can be determined as described in Altschul et al. (1997) Nucleic Acids Res., 25:3389-3402. Nonlimiting examples of low stringency and high stringency hybridization conditions are provided in subsequent sections.

The terms “specific interaction,” or “specifically binds,” or the like, mean that two molecules form a complex that is relatively stable under physiologic conditions. Specific binding is characterized by a high affinity and a low to moderate capacity. Nonspecific binding usually has a low affinity with a moderate to high capacity. Typically, the binding is considered specific when the affinity constant Kₐ is higher than 10⁶M⁻¹ or preferably higher than 10⁸M⁻¹. If necessary, nonspecific binding can be reduced without substantially affecting specific binding by varying the binding conditions. Such conditions are known in the art, and a skilled artisan using routine techniques can select appropriate conditions. The conditions are usually defined in terms of concentration of antibodies, ionic strength of the solution, temperature, time allowed for binding, concentration of unrelated molecules (e.g., serum albumin, milk casein), etc.

The phrase “substantially as set out” means that a relevant amino acid sequence is at least 70%, 75%, 80%, 85%, 90%, 95%, 97%, 98%, or 99% identical to a given sequence. By way of example, such sequences may be variants derived from various species, or they may be derived from the given sequence by truncation, deletion, amino acid substitution or addition. Percent identity between two amino acid sequences is determined by standard alignment algorithms such as, for example, Basic Local Alignment Tool (BLAST) described in Altschul et al. (1990) J. Mol. Biol., 215:403-410, the algorithm of Needleman et al. (1970) J. Mol. Biol., 48:444-453, or the algorithm of Meyers et al. (1988) Comput. Appl. Biosci., 4:11-17.

The term “germinal center” as used herein refers to a secondary B cell follicle which forms after antigen immunization. The appearance of this histologic site correlates with optimal memory generation, isotype switching, somatic hypermutation and thus the affinity maturation of an antibody response, germinal center has been well characterized in the art.

The terms “marginal zone” refers to histologically described compartments of the secondary lymphoid tissues comprised primarily of marginal zone macrophages (MZM), metallophilic macrophages (MM), marginal zone (MZ) B cells and reticular cells, and also T cells and dendritic cells. The arterial blood stream opens into the marginal sinuses thus giving antigens direct access to these cells and promoting cellular reactions to antigens at this site.

The term “LT pathway” refers to interrelated molecular events occurring in vivo as a result of the binding of LTBR and its functional ligand LT. The LT pathway is reviewed in U.S. Pat. No. 6,403,087 and in Fu et al. (1999) Ann. Rev. Immunol., 17: 399; Ruddle (1999) Immunol. Res., 19:119; Luther et al. (2000) Immunity, 12:471; and Weyand et al. (2001) Am. J. Pathol., 159:787. Surface lymphotoxin (LT) is composed of a heteromeric complex of the LT-α and LT-β, which binds uniquely to LTBR (Ware et al. (1995) Curr. Topics in Microbiol. and Immunol., 198:175). There also exists a second ligand, LIGHT, which binds not only to LTBR but also to an additional receptor called herpes virus entry mediator (HVEM). The term “LTBR ligand,” or “LT,” unless otherwise indicated, refers to the LT-α/β heterotrimer, LT-β, LT-α, and LIGHT. The term “LTBR ligand binding domain” refers to the portion or portions of LTBR which are involved in specific recognition of and interaction with LT.

The term “mammal” refers to any animal classified as such, including humans.

The term “BAFF-transgenic animal” refers to a mammal that has been genetically modified to overexpress BAFF or a functionally equivalent fragment thereof. Illustrative methods of making BAFF-transgenic animals are described later in this disclosure.

LTBR is a member of the tumor necrosis factor (TNF) family, which plays a crucial role in lymphoid organogenesis by signaling throughout its functional ligand LT. The receptor is expressed on a wide range of cell types, e.g., fibroblasts and monocytes; the ligand is expressed only on activated T, B, and NK cells. The LT pathway plays crucial roles both in the development of the secondary lymphoid system and in the establishment of ectopic organized lymphoid structures in chronically inflamed sites (Fu et al. (1999) Ann. Rev. Immunol., 17:399; Ruddle (1999) Immunol. Res., 19:119; Luther et al. (2000) Immunity, 12:471; and Weyand et al. (2001) Am. J. Pathol., 159:787).

This invention is based in part on the discovery that inhibition of the lymphotoxin (LT) pathway using a soluble form of LT-β receptor (LTBR) leads to amelioration of lupus-like disease in BAFF-transgenic mice. The invention is further based in part on the discovery that renal dysfunction in BAFF-transgenic mice is associated with accumulation of IgA and IgG immune complexes in the kidneys.

The invention also describes methods for treating or preventing glomerulonephritis comprising administering inhibitors of the LT pathway. In one embodiment, LT pathway inhibitors are administered for the treatment or prevention of glomerulonephritis associated with an autoimmune disease. The term “glomerulonephritis” refers to a renal dysfunction characterized by inflammation of the glomeruli of the kidney. Glomerulonephritis may be caused by or associated with an autoimmune condition, such as insulin dependent diabetes mellitus, multiple sclerosis, systemic lupus erythematosus (SLE), Sjogren's syndrome, scleroderma, polymyositis, chronic active hepatitis, mixed connective tissue disease, primary biliary cirrhosis, pernicious anemia, autoimmune thyroiditis, idiopathic Addison's disease, vitiligo, gluten-sensitive enteropathy, Graves' disease, myasthenia gravis, autoimmune neutropenia, idiopathic thrombocytopenia purpura, rheumatoid arthritis, cirrhosis, pemphigus vulgaris, autoimmune infertility, Goodpasture's disease, bullous pemphigoid, discoid lupus, ulcerative colitis, or dense deposit disease, In one embodiment of the invention, LTBR-Ig fusion proteins are used to treat or prevent glomerulonephritis

The invention provides methods for treating or preventing immunological disorders, including diseases caused by dysregulated production of immunoglobulins by B cells, including dysregulated production of IgA or IgG. In some embodiments, the methods may be used to prevent or to treat pathologies associated with renal dysfunction accompanied by immunoglobulin deposits.

In certain embodiments, compositions used in the methods of the invention comprise inhibitors of the LT pathway. In some embodiments, inhibitors are proteinaceous, i.e., they comprise amino acids linked by peptide bonds. Proteinaceous LT pathway inhibitors include but are not limited to soluble forms of LTBR, including LTBR-Ig, antibodies against LTBR, and antibodies against the LTBR ligand, LT. In other embodiments, compositions used in the methods of the invention comprise nonproteinaceous inhibitors of the LT pathway, such as nucleic acids, small molecule inhibitors, LT mimetics, etc. LTBR and LT, their fragments or other derivatives, or analogs thereof, may be used to generate antibodies that specifically bind LTBR or LT. In the production of antibodies, screening for the desired antibody can be accomplished by techniques known in the art, most commonly, by ELISA or FACS (for examples of assays, see, e.g., Rooney (2000) Methods Enzymol., 322:345-363).

Antibodies can be made, for example, by traditional hybridoma techniques (Kohler and Milstein (1975) Nature, 256:495-499), recombinant DNA methods (U.S. Pat. No. 4,816,567), or phage display techniques using antibody libraries (Clackson et al. (1991) Nature, 352:624-628; Marks et al. (1991) J. Mol. Biol., 222:581-597). For various other antibody production techniques, see, e.g., Antibodies: A Laboratory Manual, eds. Harlow et al., Cold Spring Harbor Laboratory, 1988. In one embodiment, antibodies used in the methods of the invention are directed against at least a part of the extracellular portion of human LTBP, In another embodiment, antibodies are capable of inhibiting the LT pathway, e.g., by blocking the binding of LT to LTBR. In another embodiment the antibody is fully human. Examples of antibodies to LTBR include but are not limited to the BDA8 monoclonal antibody directed against human LTBR that blocks the interaction between soluble LT and LTBR, and the monoclonal B9 antibody directed against human LT that blocks the interaction between cell surface LT-α/β and LTBR-Ig as described in U.S. Pat. Nos. 5,925,351 and 6,403,087, incorporated by reference herein. Antibodies to LTBR are also described in U.S. Pat. No. 6,312,691, incorporated by reference herein.

In some embodiments, the methods involve a use of soluble forms of the LTBR (e.g., LTBR-Ig fusion polypeptides) that bind LT thereby inhibiting the LT pathway in vivo. In particular, the presently disclosed soluble forms of LTBR inhibit the endogenous LTBR activity associated with dysregulated production of immunoglobulins by B cell, including dysregulated production of IgA or IgG. In some embodiments, LTBR-Ig possesses pharmacokinetic properties that make it suitable for therapeutic use, e.g., sufficiently long circulatory half-life and/or acceptable protection from proteolytic degradation.

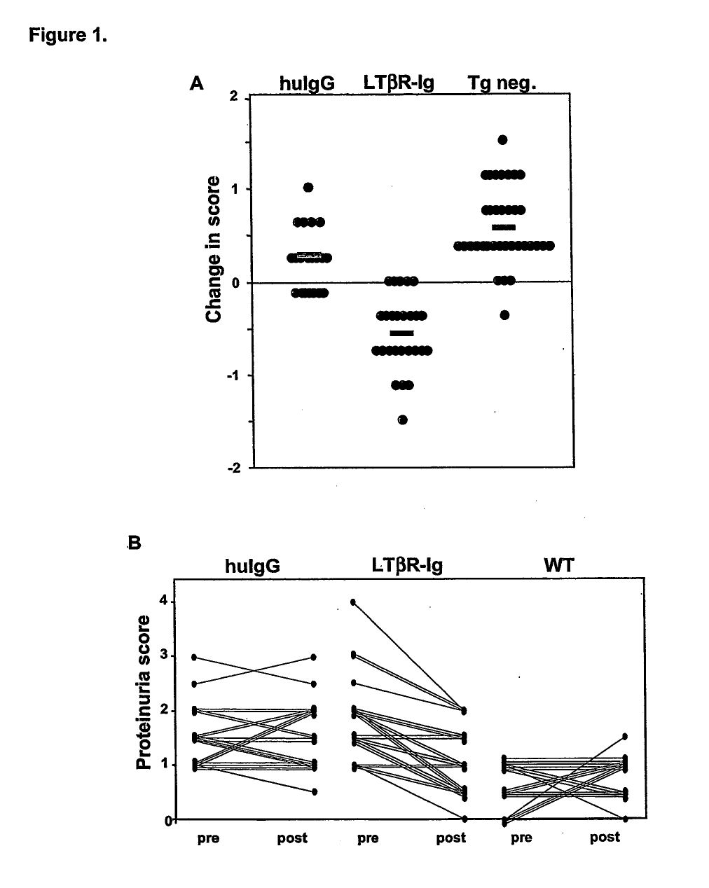

The sequence of the extracellular portion of human LTBR is set forth in SEQ ID NO: 1 and is shown in FIG. 1 of U.S. Pat. No. 5,925,351. Using the sequence information in SEQ ID NO: 1 and recombinant DNA techniques well known in the art, functional fragments encoding the LTBR ligand binding domain can be cloned into a vector and expressed in an appropriate host to produce a soluble LTBR molecule. Soluble LTBR molecules that can compete with native LTB receptors for LT ligand binding according to the assays described herein and in U.S. Pat. No. 6,403,087, are selected as LTBR blocking agents.

A soluble LTBR comprising amino acid sequences selected from those described in SEQ ID NO: 1 may be attached to one or more heterologous protein domains (“fusion domain”) to increase the in vivo stability of the receptor fusion protein, or to modulate its biological activity or localization. In one embodiment, stable plasma proteins—which typically have a half-life greater than 20 hours in the circulation—are used to construct the receptor fusion proteins. Such plasma proteins are include but are not limited to: immunoglobulins, serum albumin, lipoproteins, apolipoproteins and transferrin. Sequences that can target the soluble LTBR molecule to a particular cell or tissue type may also be attached to the LTBR ligand binding domain to create a specifically-localized soluble LTBR fusion protein.

All or a functional portion of the LTBR extracellular region (SEQ ID NO: 1) comprising the LTBR ligand binding domain may be fused to an immunoglobulin constant region like the Fc domain of a human IgG1 heavy chain (Browning et al., J. Immunol., 154, pp. 33-46 (1995)). Soluble receptor-IgG fusion proteins are common immunological reagents, and methods for their construction are known in the art (see e.g., U.S. Pat. No. 5,225,538, incorporated herein by reference).

Accordingly, in one embodiment, LTBR-Ig used in the methods of the invention comprise (a) a first amino acid sequence derived from the extracellular domain of LTBR and (b) a second amino acid sequence derived from the constant region of an antibody.

The first amino acid sequence is derived from all or a portion of the LTBR extracellular domain and is capable of binding LT specifically. The amino acid sequence of the extracellular domain of human LTBR is set out in SEQ ID NO: 1. In certain embodiments, the first amino acid sequence is identical to or is substantially as set out in SEQ ID NO:1 . Such a sequence can be truncated so long as the truncated sequence retains the ability to specifically bind LT. In some other embodiments, the first amino acid sequence comprises at least 20, 30, 40, 50, 60, 70, 80, 90, 100, 120, 140, 160, or 180 contiguous amino acids of SEQ ID NO:1.

The second amino acid sequence is derived from the constant region of an antibody, particularly the Fc portion, or is a mutation of such a sequence. In some embodiments, the second amino acid sequence is derived from the Fc portion of an IgG. In related embodiments, the Fc portion is derived from IgG that is IgG₁, IgG₄, or another IgG isotype. In a particular embodiment, the second amino acid sequence comprises the Fc portion of human IgG₁, wherein the Fc is modified to minimize the effector function. Such modifications include changing specific amino acid residues which might alter an effector function such as Fc receptor binding (Lund et al. (1991) J. Immun. 147:2657-2662 and Morgan et al. (1995) Immunology 86:319-324), or changing the species from which the constant region is derived. Antibodies may have mutations in the C_{H}2 region of the heavy chain that reduce effector function, i.e., Fc receptor binding and complement activation. For example, antibodies may have mutations such as those described in U.S. Patent Nos. 5,624,821 and 5,648,260. In the IgG₁ or IgG₂ heavy chain, for example, such mutations may be made at amino acid residues corresponding to amino acids 234 and 237 in the full-length sequence of IgG₁ or IgG₂. Antibodies may also have mutations that stabilize the disulfide bond between the two heavy chains of an immunoglobulin, such as mutations in the hinge region of IgG₄, as disclosed in Angal et al. (1993) Mol. Immunol. 30:105-108.

The Fc domains of antibodies belonging to different Ig classes or subclasses can activate diverse secondary effector functions. Activation occurs when the Fc domain is bound by a cognate Fc receptor. Secondary effector functions include the ability to activate the complement system, to cross the placenta, and to bind various microbial proteins. The properties of the different classes and subclasses of immunoglobulins are described in Roitt et al., Immunology, p. 4.8 (Mosby-Year Book Europe Ltd., 3d ed. 1993).

The complement enzyme cascade can be activated by the Fc domains of antigpri-hound IgG1, IgG3 and IgM antibodies. The Fc domain of IgG2 appears to be less effective, and the Fc domains of IgG4, IgA, IgD and IgE are ineffective at activating complement. Thus one can select a Fc domain based on whether its associated secondary effector functions are desirable for the particular immune response or disease being treated with the LT-.beta.-R-Fc fusion protein.

In certain embodiments, the second amino acid sequence is linked to the C-terminus or the N-terminus of the first amino acid sequence, with or without being linked by a linker sequence. The exact length and sequence of the linker and its orientation relative to the linked sequences may vary. The linker may, for example, comprise one or more Gly-Ser. The linker may be 2, 10,20, 30, or more amino acid long and is selected based on properties desired such as solubility, length and steric separation, immogenicity, etc. It will be understood by one of ordinary skill in the art that certain amino acids in a sequence of any protein may be substituted for other amino acids without adversely affecting the activity of the protein. It is thus contemplated that various changes may be made in the amino acid sequences of the proteinaceous LT pathway inhibitors of the invention, or DNA sequences encoding therefor without appreciable loss of their biological activity or utility.

The use of derivatives and analogs related to LTBR are within the scope of the present invention. In a specific embodiment, the derivative or analog is functionally active, i.e., capable of exhibiting one or more activities associated with the LT-binding domain of the wild-type LTBR, e.g., as set out in SEQ ID NO: 1. Derivatives or analogs that retain this binding, or inhibit the LT pathway, can be tested for the desired activity by procedures known in the art, including but not limited to the assays described in Rooney (2000) Methods Enzymol., 322:345-363, U.S. Pat. No. 6,403,087, or the Examples.

Derivatives of LTBR-Ig, LTBR antibodies, or LT antibodies can be made by altering their amino acids sequences by substitutions, additions, and/or deletions/truncations that result in functionally equivalent molecules. Due to the degeneracy of nucleotide codons, other DNA sequences that encode substantially the same amino acid sequence may be used in the practice of the present invention. These include but are not limited to nucleotide sequences that are altered by the substitution of different codons that encode a functionally equivalent amino acid residue within the sequence, thus producing a “silent” change. For example, the nonpolar amino acids include alanine, leucine, isoleucine, valine, proline, phenylalanine, typtophan, and methionine. The polar neutral amino acids include glycine, serine, threonine, cysteine, tyrosine, asparagine, and glutamine. The positively charged (basic) amino acids include arginine, lysine, and histidine. The negatively charged (acidic) amino acids include aspartic acid and glutamic acid. Substitutes for an amino acid within the sequence may be selected from other members of the class to which the amino acid belongs (see Table 1). Furthermore, any native residue in the polypeptide may also be substituted with alanine (see, e.g., MacLennan et al. (1998) Acta Physiol. Scand. Suppl. 643:55-67; Sasaki et al. (1998) Adv. Biophys. 35:1-24).

The LTBR derivatives and analogs of the invention can be produced by various techniques well known in the art, including recombinant and synthetic methods (Maniatis (1990) Molecular Cloning, A Laboratory Manual, 2nd ed., Cold Spring Harbor Laboratory, Cold Spring Harbor, N.Y., and Bodansky et al. (1995) The Practice of Peptide Synthesis, 2nd ed., Spring Verlag, Berlin, Germany).

TABLE 1

| Original | Exemplary | Typical |
|---|---|---|
| Residues | Substitutions | Substitutions |
| Ala (A) | Val, Leu, Ile | Val |
| Arg (R) | Lys, Gln, Asn | Lys |
| Asn (N) | Gln | Gln |
| Asp (D) | Glu | Glu |
| Cys (C) | Ser, Ala | Ser |
| Gln (Q) | Asn | Asn |
| Gly (G) | Pro, Ala | Ala |
| His (H) | Asn, Gln, Lys, Arg | Arg |
| Ile (I) | Leu, Val, Met, Ala, Phe, Norleucine | Leu |
| Leu (L) | Norleucine, Ile, Val, Met, Ala, Phe | Ile |
| Lys (K) | Arg, 1,4-Diamino-butyric Acid, Gln, Asn | Arg |
| Met (M) | Leu, Phe, Ile | Leu |
| Phe (F) | Leu, Val, Ile, Ala, Tyr | Leu |
| Pro (P) | Ala | Gly |
| Ser (S) | Thr, Ala, Cys | Thr |
| Thr (T) | Ser | Ser |
| Trp (W) | Tyr, Phe | Tyr |
| Tyr (Y) | Trp, Phe, Thr, Ser | Phe |
| Val (V) | Ile, Met, Leu, Phe, Ala, Norleucine | Leu |

In certain embodiments, additional fusions of any of LTBR-Ig of the invention to amino acid sequences derived from other proteins may be constructed for use in the methods of the invention. Desirable fusion sequences may be derived from proteins having biological activity different from that of LTBR, for example, cytokines, growth and differentiation factors, enzymes, hormones, other receptor components, etc. Also, LTBR-Ig may be chemically coupled, or conjugated, to other proteins and pharmaceutical agents. Such modifications may be designed to alter the pharmacokinetics and/or biodistribution of the resultant composition. The LTBR-Ig and antibodies of the invention may also be glycosylated, pegylated, or linked to another nonproteinaceous polymer, e.g., polyethylene glycol, polypropylene glycol, or polyoxyalkylenes, in the manner set forth in U.S. Pat. Nos. 4,640,835; 4,496,689; 4,301,144; 4,670,417; 4,791,192; or 4,179,337. The LTBR-Ig and antibodies maybe chemically modified by covalent conjugation to a polymer to increase their circulating half-life, for example. Exemplary polymers, and methods to attach them to peptides, are also shown in U.S. Pat. Nos. 4,766,106; 4,179,337; 4,495,285; and 4,609,546.

The LTBR-Ig and antibodies used in the methods of the invention may also be tagged with a detectable or functional label. Detectable labels include radiolabels such as ¹³¹I or ⁹⁹Tc, which may be attached using conventional chemistry. Detectable labels further include enzyme labels, e.g., horseradish peroxidase or alkaline phosphatase and detectable moieties such as biotin or avidin.

In some embodiments, the methods of the invention comprise administration of nucleic acids or polypeptides encoded by such nucleic acids, where the nucleotide sequence is selected from: (a) a nucleotide sequence encoding the polypeptide of SEQ ID NO: 1; and (b) a nucleic acid that is at least 100, 200, 300, 400, or 500 nucleotides long and is capable of hybridizing to the nucleic acid of (a) under defined conditions; wherein the expression product of the nucleic acid is capable of inhibiting immunoglobulin secretion by B cells. In one embodiment, the defined conditions are low stringency conditions. In another embodiment, the defined conditions are moderate stringency conditions. In yet another embodiment, the defined conditions are high stringency conditions.

Appropriate hybridization conditions can be selected by those skilled in the art with minimal experimentation as exemplified in Ausubel et al. (1995), Current Protocols in Molecular Biology, John Wiley & Sons, sections 2, 4, and 6. Additionally, stringent conditions are described in Sambrook et al. (1989) Molecular Cloning: A Laboratory Manual, 2nd ed., Cold Spring Harbor Press, chapters 7, 9, and 11. A nonlimiting example of defined conditions of low stringency is as follows. Filters containing DNA are pretreated for 6 h at 40° C. in a solution containing 35% formamide, 5×SSC, 50 mM Tris-HCl (pH 7.5), 5 mM EDTA, 0.1% PVP, 0.1% Ficoll, 1% BSA, and 500 μg/ml denatured salmon sperm DNA. Hybridizations are carried out in the same solution with the following modifications: 0.02% PVP, 0.02% Ficoll, 0.2% BSA, 100 μg/ml salmon sperm DNA, 10% (wt/vol) dextran sulfate, and 5-20×10^{6 32}P-labeled probe is used. Filters are incubated in hybridization mixture for 18-20 h at 40° C, and then washed for 1.5 h at 55° C. in a solution containing 2×SSC, 25 mM Tris-HCl (pH 7.4), 5 mM EDTA, and 0.1% SDS. The wash solution is replaced with fresh solution and incubated an additional 1.5 h at 60° C. Filters are blotted dry and exposed for autoradiography. Other conditions of low stringency well known in the art may be used (e.g., as employed for cross-species hybridizations).

A nonlimiting example of defined conditions of high stringency is as follows. Prehybridization of filters containing DNA is carried out for 8 h to overnight at 65° C. in buffer composed of 6×SSC, 50 mM Tris-HCl (pH 7.5), 1 mM EDTA, 0.02% PVP, 0.02% Ficoll, 0.02% BSA, and 500 μg/ml denatured salmon sperm DNA. Filters are hybridized for 48 h at 65° C. in the prehybridization mixture containing 100 μg/ml denatured salmon sperm DNA and 5-20×10⁶ cpm of ³²P-labeled probe. Washing of filters is done at 37° C. for 1 h in a solution containing 2×SSC, 0.01% PVP, 0.01% Ficoll, and 0.01% BSA. This is followed by awash in 0.1×SSC at 50° C. for 45 minutes.

Systems for cloning and expression of a polypeptide in a variety of different host cells are well known. Suitable host cells include bacteria, mammalian cells, and yeast and baculovirus systems. Mammalian cell lines available in the art for expression of a heterologous polypeptide include Chinese hamster ovary cells, HeLa cells, baby hamster kidney cells, NSO mouse melanoma cells and many others. A common bacterial host is E. coli. For other cells suitable for producing, e.g., LTBR-Ig, see Gene Expression Systems, eds. Fernandez et al., Academic Press, 1999.

Suitable vectors can be chosen or constructed, containing appropriate regulatory sequences, including promoter sequences, terminator sequences, polyadenylation sequences, enhancer sequences, marker genes and other sequences as appropriate. Vectors may be plasmids or viral, e.g., phage, or phagernid, as appropriate. For further details see, e.g., Molecular Cloning: A Laboratory Manual, Sambrook et al., 2nd ed., Cold Spring Harbor Laboratory Press, 1989. Many known techniques and protocols for manipulation of nucleic acid, for example, in preparation of nucleic acid constructs, mutagenesis, sequencing, introduction of DNA into cells and gene expression, and analysis of proteins, are described in detail in Current Protocols in Molecular Biology, eds. Ausubel et al., 2nd ed., John Wiley & Sons, 1992.

Following expression, LTBR-Ig is isolated and/or purified (exemplary procedures for expression and purification of LTBR-Ig, LT, including LT-α, LT-β, and LT-α/β, are described in Rooney (2000) Methods Enzymol., 322:345-363). Specific LTBR-Ig and their encoding nucleic acid molecules and vectors according to the present invention may be obtained, isolated and/or purified, e.g., from their natural environment, in substantially pure or homogeneous form, or in the case of nucleic acid, free or substantially free of nucleic acid or genes origin other than the sequence encoding a polypeptide with the required function.

The invention provides methods for treatment or prevention of various diseases and disorders by administration of a therapeutic compound (“therapeutic”). Suitable therapeutics include but are not limited to: LTBR, analogs and derivatives (including fragments) thereof; nucleic acids encoding the LTBR proteins, analogs, or derivatives; LTBR antisense nucleic acids, LTBR antibodies, LT antibodies, and other LTBR/LT antagonists.

Examples of immunological disorders susceptible to treatment by the methods of the invention include but are not limited to autoimmune conditions such as insulin dependent diabetes mellitus, multiple sclerosis, systemic lupus erythematosus, Sjogren's syndrome, scleroderma, polymyositis, chronic active hepatitis, mixed connective tissue disease, primary biliary cirrhosis, pernicious anemia, autoimmune thyroiditis, idiopathic Addison's disease, vitiligo, gluten-sensitive enteropathy, Graves' disease, myasthenia gravis, autoimmune neutropenia, idiopathic thrombocytopenia purpura, rheumatoid artritis, cirrhosis, pemphigus vulgaris, autoimmune infertility, Goodpasture's disease, bullous pemphigoid, discoid lupus, ulcerative colitis, or dense deposit disease. Immunological disorders in which production of immungolublins by B cells is dysregulated, including dysregulated production of immunoglobulins, e.g., IgA or IgG, are treated or prevented by administration of a therapeutic disclosed herein. In certain embodiments, the B cells are IgA-overproducing B-1 cells. In some embodiments, the disease is characterized by elevated (relative to normal or desired) levels auto-antibodies. In some other embodiments, the dysregulated production of immunoglobulins results in pathological deposits of IgA in kidneys. Such disorders include but are not limited to systemic lupus erythematosus, Sjogren's syndrome, and rheumatoid arthritis, insulin dependent diabetes mellitus (IDDM), chronic hepatitis,

Henoch-Schonlein purpura (HSP), IgA nephropathy (Berger's disease) as well as any other disease, the clinical manifestations of which includes the presence of macroscopic or microscopic hematuria and renal IgA deposits. In some other embodiments, the methods of the invention are employed to treat autoimmune disorders that have a GC-independent mechanism. In yet other embodiments, the methods are utilized to treat a disease characterized by an elevated (relative to normal or desired) level of MZ B cells such as, e.g., Sjogren's syndrome. In still another embodiment, LT pathway inhibitors are used to treat subjects with glomerulonephritis, including, for example, glomerulonephritis associated with an autoimmune condition.

In specific embodiments, therapeutics that inhibit LTBR function are administered in: (1) diseases or disorders involving elevated (i.e., relative to normal or desired) levels of expression of LTBR or LT or elevated LTBR or LT functional activity, or (2) diseases or disorders where in vitro or in vivo assays indicate the utility of LT pathway inhibitor administration. The elevated level of expression or activity can be readily detected, e.g., by obtaining a biological sample from a patient, e.g., a urine sample, and assaying it for the presence of macroscopic or microscopic hematuria. Many methods standard in the art can be employed, including but not limited to kinase assays, immunoassays to detect and/or visualize LTBR protein (e.g., Western blot, immunoprecipitation followed by SDS-PAGE, immunocytochemistry, etc.) and/or hybridization assays (e.g., Northern assays, dot blots, in situ hybridization, RT-PCR, etc.).

One embodiment of the invention provides assay for identifying inhibitors of the LT pathway effective as therapeutics for treatment of autoimmune disorders, including diseases associated with Ig deposits in kidneys. In this screening assay, a first binding mixture is formed by combining an LTBR-Ig fusion polypeptide and a ligand, e.g., LT; and the amount of binding in the first binding mixture (M₀) is measured. A second binding mixture is also formed by combining the LTBR-Ig fusion polypeptide, the ligand, and the compound or agent to be screened, and the amount of binding in the second binding mixture (M₁) is measured. The amounts of binding in the first and second binding mixtures are then compared, for example, by calculating the M₁/M₀ ratio. The compound or agent is considered to be capable of inhibiting Ig-mediated kidney disease if a decrease in binding in the second binding mixture as compared to the first binding mixture is observed. The formulation and optimization of binding mixtures is within the level of skill in the art, such binding mixtures may also contain buffers and salts necessary to enhance or to optimize binding, and additional control assays may be included in the screening assay of the invention. Compounds found to reduce LTBR/LT binding by at least about 10% (i.e., M₁/M₀<0.9), preferably greater than about 30% or may thus be identified and then, if desired, secondarily screened for the capacity to ameliorate an autoimmune disorder in other assays or animal models as described below. The strength of the binding between a receptor and ligand can be measured using, for example, an enzyme-linked immunoadsorption assay (ELISA), radio-immunoassay (RIA), surface plasmon resonance-based technology (e.g., Biacore), all of which are techniques well known in the art.

LT pathway inhibitors have been found to alleviate renal dysfunction in BAFF-transgenic animals without complicating developmental defects observed in the knockout model. Thus, such animal models can be used in an assay for identifying and/or testing efficacy of a therapeutic compound for disorders involving dysregulated production of immunoglobulins by B cells, including dysregulated production of IgA or IgG. Certain aspects of immune-related renal pathology are observed in BAFF-transgenic animals. BAFF-transgenic mice begin to develop a lupus-like syndrome at 6 months of age and older as assessed by scoring protein levels in the urine. The increase in Ig titers in BAFF-transgenic animals is most marked for the IgA subclass, and from collected data there appears to be more IgA-secreting cells than IgG-secreting cells in the kidney. Furthermore, injection of BAFF into mice results in a hyperresponse to pneumovax antigen, and the most dramatic isotype affected is IgA. Therefore, in certain embodiments, BAFF-transgenic animals are used to evaluate the efficacy of a compound for treatment of IgA-related immunological disorders, including IgA nephropathy or other disorders with a strong IgA component. BAFF-transgenic animals can also be used to mimic disease states in which follicular B cells and the GC reaction are dispensable. The BAFF-transgenic animal model can be used to screen for or test molecules for the ability to treat or prevent immunological disorders such as, for example, systemic lupus erythematosus (SLE), IgA nephropathy (Berger's disease), Henoch-Schonlein purpura (HSP), Sjogren's syndrome, scleroderma, polymyositis, insulin dependent diabetes mellitus, multiple sclerosis, chronic active hepatitis, mixed connective tissue disease, primary biliary cirrhosis, pernicious anemia, autoimmune thyroiditis, idiopathic Addison's disease, vitiligo, gluten-sensitive enteropathy, Graves' disease, myasthenia gravis, autoimmune neutropenia, idiopathic thrombocytopenia purpura, rheumatoid arthritis, cirrhosis, pemphigus vulgaris, autoimmune infertility, Goodpasture's disease, bullous pemphigoid, discoid lupus, ulcerative colitis, or dense deposit disease.

BAFF-transgenic mice express full-length murine BAFF under the control of liver-specific regulatory sequences and are generated as previously described (Mackay et al. (1999) J. Exp. Med. 190:1697). Mice. can be generated through ongoing colony expansion by back-crossing transgenic males to C57BL/6 females. Transgenic status can be determined by performing PCR on DNA collected from tail tips. For transgenic methods see, generally, Transgenic Mouse Methods and Protocols, eds. Hofker et al., Humana Press, 2002. In brief, BAFF-transgenic mice were generated as follows. A PCR fragment encoding full-length murine BAFF was generated by reverse transcription-PCR using previously described sequence information (Scheider et al. (1999) J. Exp. Med., 189:1747-1756). First strand cDNA was synthesized from mouse lung polyA+ (Clontech) using oligo dT according to the manufacturer's protocol (GIBCO BRL). The PCR reaction contained 1×pfu buffer (Stratagene Inc.), 0.2 mM dNTPs, 10% DMSO, 12.5 pM primers, 5 units pfu enzyme (Stratagene Inc.), and the following primers Not1 restriction sites

(SEQ ID NO:2)

5′-TAAGAATGCGGCCGCGGAATGGATGAGTCTGCAAA-3′

(SEQ ID NO:3)

5′-TAAGAATGCGGCCGCGGGATCACGCACTCCAGCAA-3′.

The template was amplified for 30 cycles at 94° C. for 1 min, 54° C. for 2 min, and 72° C. for 3 min, followed by a 10-min extension at 72° C. This sequence corresponds to nucleotides 214-1171 of the GenBank file AF119383. The PCR fragment was digested with Not1 and cloned into a modified pCEP4 vector (Invitrogen Corp.). The resulting vector was then digested Xba1 to remove BAFF plus the SV40 polyA addition site sequence. This fragment was cloned into a pUC-based vector in which the promoter, a 1-kb blunt Bgl2-Not1 fragment containing the human ApoE enhancer and AAT (a-antitrypsin) previously purified from the plasmid clone 540B was further inserted at the EcoRV site. An EcoRV/Bgl2 fragment was purified from the final vector and used for the generation of transgenic mice. The injected offspring of C57BL/6J female X DBA/2J male F1 (BDF1) mice were backcrossed onto C57BL/6 mice. Techniques of microinjection and generation of transgenic mice have been previously described (Mcknights et al. (1983) Cell, 34:335-341).

One of skill in the art will appreciate that a compound may be optionally tested in at least one additional animal model (see, generally, Immunologic Defects in Laboratory Animals, eds. Gershwin et al., Plenum Press, 1981), for example, such as the following: the SWR X NZB (SNF1) transgenic mouse model (Uner et al. (1998) J. Autoimmune. 11(3):233-240), the KRN transgenic mouse (K/BxN) model (Ji et al. (1999) Immunol. Rev. 169:139); NZB X NZW (B/W) mice, a model for SLE (Riemekasten et al. (2001) Arthritis Rheum., 44(10):2435-2445); experimental autoimmune encephalitis (EAE) in mouse, a model for multiple sclerosis (Tuohy et al. (1988) J. Immunol. 141:1126-1130, Sobel et al. (1984) J. Immunol. 132:2393-2401, and Traugott, Cell Immunol. (1989) 119:114-129); the NOD mouse model of diabetes (Baxter et al. (1991) Autoimmunity, 9(1):61-67), etc.).

In certain embodiments, compounds to be tested comprise inhibitors of the LT pathway such as, e.g., soluble forms of LTBR (e.g., LTBR-Ig), antibodies against LTBR, and antibodies against the LTBR ligand, LT; analogs, derivatives, and fragments thereof; their encoding and antisense nucleic acids (and complementary and homologous sequences thereof). In an illustrative embodiment, mice are injected intraperitoneally with about 1 μg to about 1 mg, preferably about 10 μg to about 500 μg, or more preferably about 100 μg of LTBR-Ig or an Ig control for a 5 week period. Kidney function is assessed by at least one of the following: proteinurea, immunohistologic analysis, spleen weight, splenocyte count, follicular B cell count, Marginal Zone B cell count (e.g., percentage of B220+ splenocytes), ratio B-1a to B-2b cells, plasma cell count and IgA+plasma cell count, and frequency IgA or IgG secretors in spleen, bone marrow, or kidney, etc. Sera may be also evaluated for the presence of auto-anti-nuclear antibodies (ANA), which bind to cellular nuclear antigens are known as a classic marker of systemic rheumatic diseases including SLE, Sjogren's syndrome, mixed connective tissue diseases (MCID), and systemic sclerosis.

Preliminary doses as, for example, determined according to animal tests, and the scaling of dosages for human administration is performed according to art-accepted practices. Toxicity and therapeutic efficacy can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Compositions that exhibit large therapeutic indices are preferable.

The therapeutically effective dose can be estimated initially from cell culture assays. A dose may be formulated in animal models to achieve a circulating plasma concentration range that includes the IC₅₀ (i.e., the concentration of the therapeutic which achieves a half-maximal inhibition of symptoms) as determined in cell culture assays or animal models. Levels in plasma may be measured, for example, by high performance liquid chromatography. The effects of any particular dosage can be monitored by a suitable bioassay. Examples of dosages are: about 0.1×IC₅₀, about 0.5×IC₅₀, about 1×IC₅₀, about 5×IC₅₀, 10×IC₅₀, about 50×IC₅₀, and about 100×IC_{50.}

The data obtained from the cell culture assays or animal studies can be used in formulating a range of dosage for use in humans. Therapeutically effective dosages achieved in one animal model can be converted for use in another animal, including humans, using conversion factors known in the art (see, e.g., Freireich et al. (1966) Cancer Chemother. Reports, 50(4):219-244 and Table 2 for Equivalent Surface Area Dosage Factors).

TABLE 2

| | To: | | | | |
|---|---|---|---|---|---|
| | Mouse | Rat | Monkey | Dog | Human |
| From: | (20 g) | (150 g) | (3.5 kg) | (8 kg) | (60 kg) |
| Mouse | 1 | 1/2 | 1/4 | 1/6 | 1/12 |
| Rat | 2 | 1 | 1/2 | 1/4 | 1/7 |
| Monkey | 4 | 2 | 1 | 3/5 | 1/3 |
| Dog | 6 | 4 | 3/5 | 1 | 1/2 |
| Human | 12 | 7 | 3 | 2 | 1 |

The dosage of such compounds lies preferably within a range of circulating concentrations that include the ED₅₀ with little or no toxicity. The dosage may vary within this range depending upon the dosage form employed and the route of administration utilized. Generally, a therapeutically effective amount may vary with the subject's age, condition, and sex, as well as the severity of the medical condition in the subject. The dosage may be determined by a physician and adjusted, as necessary, to suit observed effects of the treatment. Generally, the compositions are administered so that LT pathway inhibitors are given at a dose approximately: from 1 μg/kg to 20 mg/kg, from 1 μg/kg to 10 mg/kg, from 1 μg/kg to 1 mg/kg, from 10 μg/kg to 1 mg/kg, from 10 μg/kg to 100 μg/kg, from 100 μg to 1 mg/kg, and from 500 μg/kg to 1 mg/kg. The compositions may be given as a bolus dose, to maximize the circulating levels for the greatest length of time after the dose. Continuous infusion may also be used after the bolus dose.

In some embodiments, compositions used in the methods of the invention comprise one or more inhibitors of the LT pathway and a pharmaceutically acceptable excipient. As used herein, the phrase “pharmaceutically acceptable excipient” refers to any and all solvents, dispersion media, coatings, antibacterial and antifungal agents, isotonic and absorption delaying agents, and the like, that are compatible with pharmaceutical administration. The use of such media and agents for pharmaceutically active substances is well known in the art. The compositions may also contain other active compounds providing supplemental, additional, or enhanced therapeutic functions. The pharmaceutical compositions may also be included in a container, pack, or dispenser together with instructions for administration.

A pharmaceutical composition is formulated to be compatible with its intended route of administration. Methods to accomplish the administration are known in the art. The administration may, for example, be intravenous, intraperitoneal, intramuscular, intracavity, subcutaneous, or transdermal. It may also be possible to obtain compositions that may be administered topically or orally.

Solutions or suspensions used for intradermal or subcutaneous application typically include one or more of the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid, buffers such as acetates, citrates or phosphates; and agents for the adjustment of tonicity such as sodium chloride or dextrose. The pH can be adjusted with acids or bases, such as hydrochloric acid or sodium hydroxide. Such preparations may be enclosed in ampoules, disposable syringes, or multiple dose vials made of glass or plastic.

Pharmaceutical compositions suitable for injection include sterile aqueous solutions or dispersions and sterile powders for the extemporaneous preparation of sterile injectable solutions or dispersion. For intravenous administration, suitable carriers include physiological saline, bacteriostatic water, Cremophor EL (BASF, Parsippany, N.J.) or phosphate buffered saline (PBS). In all cases, the composition must be sterile and should be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol, and sodium chloride in the composition. Prolonged absorption of the injectable compositions can be brought about by including in the composition an agent which delays absorption, for example, aluminum monostearate and gelatin.

Oral compositions generally include an inert diluent or an edible carrier. They can be enclosed in gelatin capsules or compressed into tablets. For the purpose of oral therapeutic administration, the LT inhibitors can be incorporated with excipients and used in the form of tablets, troches, or capsules. Pharmaceutically compatible binding agents, and/or adjuvant materials can be included as part of the composition. The tablets, pills, capsules, troches, and the like can contain any of the following ingredients, or compounds of a similar nature; a binder such as microcrystalline cellulose, gum tragacanth or gelatin; an excipient such as starch or lactose, a disintegrating agent such as alginic acid, Primogel, or corn starch, a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the LT inhibitors are delivered in the form of an aerosol spray from pressured container or dispenser which contains a suitable propellant, e.g., a gas such as carbon dioxide, or a nebulizer.

Systemic administration can also be by transmucosal or transdermal means. For example, in case of LTBR-Ig and antibodies, compositions may be capable of transmission across mucous membranes (e.g., intestine, mouth, or lungs) via the FcRn receptor-mediated pathway (U.S. Pat. No. 6,030,613). Transmucosal administration can be accomplished, for example, through the use of lozenges, nasal sprays, inhalers, or suppositories. For transdermal administration, the active compounds are formulated into ointments, salves, gels, or creams as generally known in the art. For transmucosal or transdermal administration, penetrants appropriate to the barrier to be permeated are used in the formulation. Such penetrants are generally known in the art, and include, for example, detergents, bile salts, and fusidic acid derivatives.

The LT inhibitors can prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. Liposomal suspensions can also be used as pharmaceutically acceptable carriers. These can be prepared according to methods known to those skilled in the art, for example, as described in U.S. Pat. No. 4,522,811.

It is may be advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier.

Nucleic acids encoding proteinaceous LT inhibitors, such as the nucleic acids encoding all or a part of LTBR or their corresponding antisense nucleic acids, can be introduced to a cell within tissue, an organ, or an organism so that the encoded polypeptides can then be expressed. For specific protocols, see Morgan, Gene Therapy Protocols, 2nd ed., Humana Press, 2000. This methodology may be useful, for example, in evaluating effects of proteinaceous LT inhibitors on individual tissues and organs. In certain embodiments, nucleic acid encoding a proteinaceous LT inhibitor is linked to a tissue-specific expression control sequence. Those of skill in the art will recognize that specific polynucleotide sequences can be inserted into the viral or plasmid vectors that can be injected into a mammal systemically, or locally. Host cells may also be harvested, and a nucleic acid encoding a proteinaceous LT inhibitor may be transfected into such cells ex vivo for subsequent reimplantation using methods known in the art. Nucleic acids may be also transfected into a single cell embryo to create a transgenic animal as described in Gene Expression Systems, Academic Press, eds. Fernandez et al., 1999.

### EXAMPLES

### Example 1

### LTBR-Ig Treatment Ameliorates Kidney Function

BAFF-transgenic (Tg) mice expressing full-length murine BAFF under the control of liver-specific regulatory sequences were generated as previously described Mackay et al. (1999) J. Exp. Med. 190:1697). All mice used were generated through ongoing colony expansion by back-crossing Tg males to C57BL/6 females. Transgenic status was determined by performing PCR on DNA collected from tail tips. BAFF-transgenic mice begin to develop a lupus-like syndrome at 6 months of age and older as assessed by scoring protein levels in the urine.

In order to evaluate whether LTBR-Ig would be effective in treating BAFF-transgenic mice, animals that were 6 months of age or older exhibiting proteinuria (PU) scores of 1+/2+ or higher were selected for enrollment in a 5-week treatment regime. BAFF Tg mice and nontransgenic (Tg neg.) littermate controls received intraperitoneal (i.p.) injections of 100 μg LTBR-Ig or 100 μg of human IgG (huIgG) (Sandoz, Basel, Switzerland) once a week for 5 weeks. Mice were euthanized 4 days after the last injection and kidneys were harvested for histologic analysis.

In four separate experiments, LTBR-Ig treatment was found to significantly decrease proteinuria scores after the 5 week period of treatment (pretreatment PU levels versus post-treatment PU levels, P<0.0004, n=26) whereas treatment with a control matched protein failed to ameliorate kidney function (P=0.8, n=23) (FIG. 1A). The PU scores recorded from transgene negative littermates changed modestly over the treatment period, however, the scores remained below +1 (no disease) for the entire treatment regime thus rendering these fluctuations in PU score meaningless (FIG. 1B). In addition, there were no observable differences between LTBR-Ig and huIgG control treated transgene negative littermates, therefore their scores were combined.

In order to compare aspects of the BAFF-transgenic model with another lupus model, SNF1 mice were treated either therapeutically or prophylactically with LTBR-Ig. Glumerulonephritis (GN) in these animals is significantly improved with MR1 treatment, likely due to its effects on the GC reaction. Mice were treated either prior to disease onset when their proteinuria scores were approximately +1, or they were treated therapeutically when scores had reached +3/+4. In either case, LTBR-Ig treatment had no effect on proteinuria measurements or kidney pathology. There was some survival advantage when these animals were dosed with LTBR-Ig, however this is independent of GN status. Thus, although the LT pathway appears to be important in SLE-like disease in the BAFF-transgenic mice, this pathway is less important in the SNF1 model where MR1 is more efficacious.

### Example 2

### LTBR-IG Treatment Ameliorates Kidney Glomerular Damage

Kidney dysfunction as assessed by proteinuria accompanies aspects of kidney pathology. In nephritic kidneys, several pathologies may be apparent. Glomemli are seen as enlarged and hypercellular, and collagen deposits within the glomeruli can be apparent In addition, infiltrates are often observed around glomeruli and in more severely nephritic cases, proteinaceous casts can be discerned.

BAFF-Tg mice and nontransgenic (Tg neg.) controls were treated as with LTBR-Ig or huIgG as described in Example 1. Using periodic acid-Schiff (PAS) staining to evaluate collagen deposits, kidneys from 4 separate studies were evaluated.

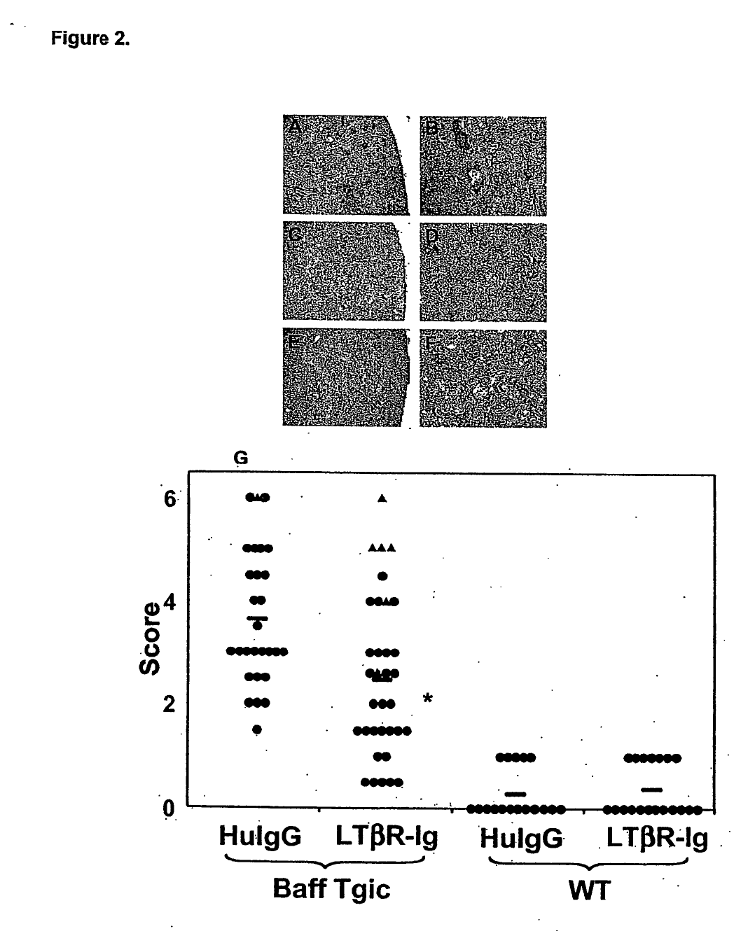

A statistically significant (P<0.002) decrease in kidney pathology was observed with LTBR-Ig treatment as compared to the huIgG control animals (FIG. 2G). The range of pathology from animal to animal was significant, and LTBR-Ig treatment did not prevent pathology in every animal. Representative histochemical samples are shown in FIGS. 2A-2F. While LTBR-Ig treatment likely prevents accelerated kidney pathology, mice with starting PU scores higher than 3 appeared to be less susceptible to the treatment and exhibited the greatest amount of kidney pathology post-treatment (FIG. 2G, triangles).

### Example 3

### LTBR-Ig Treatment Decreases Auto-Antibody Titers

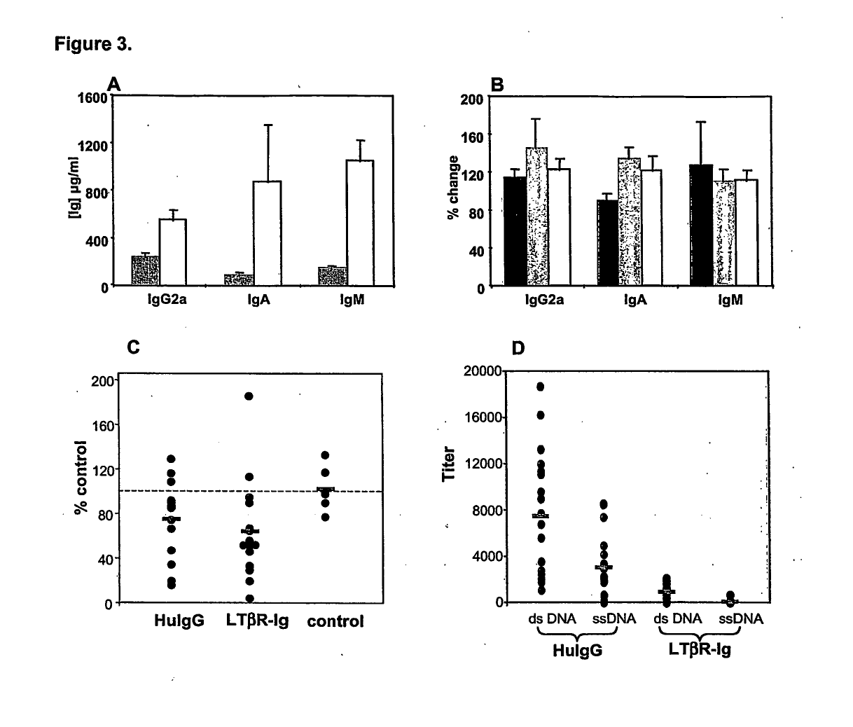

Serum Ig levels in BAFF-Tg mice have been shown to be elevated compared to non-transgenic littermates. As shown in FIG. 3A, using BAFF-Tg mice, a marked increase in both IgM (7.3 fold) and IgA (10.6 fold) titers, and a moderate increase in IgG2a titers (2.4 fold). Total IgG titers were increased by only 1.2 fold were found.

To determine if inhibition of the LT pathway in a hyper-IgA mouse would have an effect on overall IgA levels. Examination of auto-antibody titers was performed in BAFF-transgenic mice. LTBR-Ig treatment over 5 weeks did not have any impact on any Ig isotype, including IgA (FIGS. 3B and 3C). Only approximately 30% of BAFF-transgenics had evidence of anti-DNA titers, and these were very weak. Anti-chromatin titers were more reliably present in serum of transgenic mice. Treatment with LTBR-Ig resulted in a 36% decrease in anti-chromatin titers over the 5 week treatment period. However, IgG-treated transgenic mice exhibited a 25% decrease in anti-chromatin titers over the same treatment period.

Auto-antibody titers do not necessarily correlate with disease in a number of animal models. The low titers of anti-DNA antibodies in BAFF-transgenic mice was certainly inconsistent with the kidney pathology observed. Therefore, it was desirable to determine whether LTBR-Ig treatment could impact generation of these auto-antibodies in a system that more reliably results in the production of anti-DNA antibodies. A single injection of pristane has been shown to induce anti-DNA antibodies in a variety of background mouse strains, although the mechanism of action is unclear. The effect of LTBR-Ig treatment in pristane-injected mice was evaluated. 4 weeks after pristane injection into DBA-1 mice, measurable anti-double stranded (ds) DNA and anti-single stranded (ss) DNA titers were observed in the serum of these animals (FIG. 3D) whereas non-injected mice did not generate anti-DNA titers. LTBR-Ig treatment completely prevented the induction of anti-dsDNA and anti-ssDNA titers. Therefore, in a system where auto-antibody titers are more robust, these titers can be prevented by LTBR-Ig treatment.

### Example 4

### Immune Complex Deposits in the Kidneys of BAFF-Transgenic Mice

Kidney pathology associated with SLE is often accompanied by deposits of immune complexes within the glomeruli. BAFF-transgenic mice have been shown to have high levels of circulating immune complexes. These mice also exhibit evidence of immune complex deposits in the glomeruli.

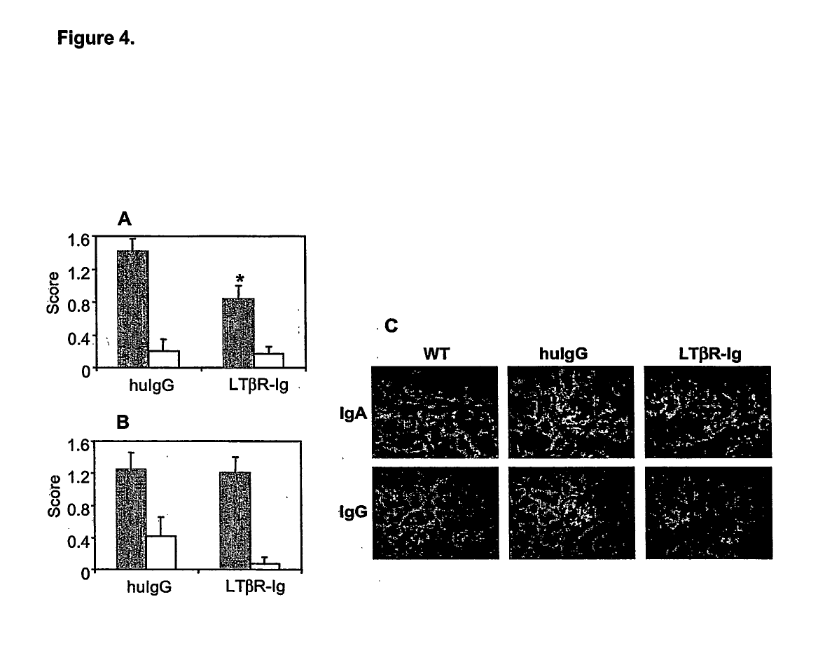

The presence of each IgA and IgG immune complexes in BAFF-transgenic kidneys was examined. Frozen kidney sections were acetone fixed, air dried and incubated with 10% Fc Block (Pharmingen, San Diego, Calif.), 5% rat serum and 5% BSA in TBS buffer to block nonspecific binding sites. Sections were incubated with a biotin-conjugated rat anti-mouse IgA-specific monoclonal antibody for 30 minutes, followed by streptavidin-488 (Molecular Probes, Eugene, Oreg.) for 30 minutes. All incubations were done at room temperature in a humidified chamber. Glomeruli were examined under UV light and scored for the presence of IgA deposits. Although titers of serum IgA were unchanged with 5 weeks of LTBR-Ig treatment, the appearance of IgA immune complexes in glomeruli of BAFF-transgenic mice was significantly decreased (P<0.01 compared to control treated BAFF-transgenics) (FIGS. 4A and 4C). No significant difference in IgG immune complex deposits was detected with treatment (FIGS. 4B and 4C), suggesting that diminishment of this disease parameter does not contribute to the efficacy observed with LTBR-Ig treatment.

### Example 5

### LTBR-Ig Treatment Reduces Marginal Zone B Cells

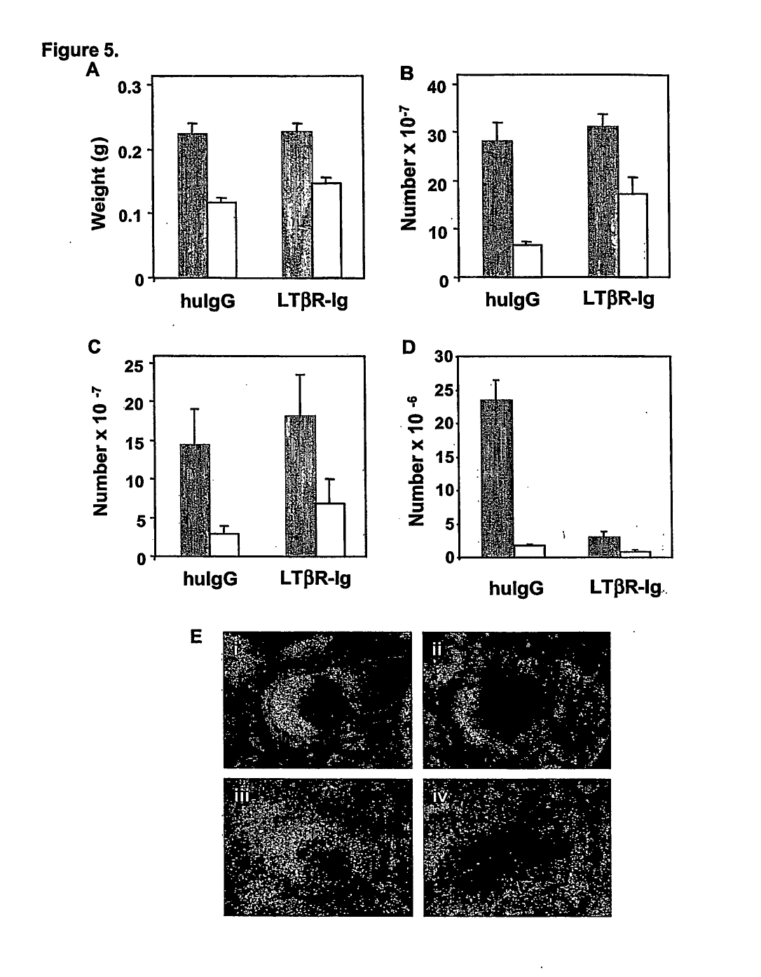

BAFF-transgenic mice exhibit an expansion of T2 transitional stage B cells, MZ B cells, mature follicular B cells, and in some cases B-1 cells. This expansion accompanies an increase in spleen weight and cellularity. Mice were treated as described in Example 1. After the 5 week treatment period, these parameters were analyzed for their ability to be modulated by LT inhibition. Spleen weights for BAFF-transgenic mice were on average 1.5-fold heavier than transgene-negative littermates and LTBR-Ig treatment had no effect on this splenomegaly (FIG. 5A). Furthermore, neither spleen cellularity nor the number of mature CD23+ follicular B cell numbers was changed with LTBR-Ig treatment (FIG. 5B), as was (FIG. 5C). MZ B cell numbers were evaluated by enumerating CD23-CD1hiCD21hi B cells. In contrast to the follicular B cell subset that was increased by approximately 4-fold in spleens of BAFF-transgenic versus non-transgenic littermates, MZ B cell numbers were increased by approximately 13-fold. In spite of this hyper-expansion, LTBR-Ig treatment dramatically reduced MZ B cells numbers compared to control treatment (P<0.00006) (FIG. 5D). Furthermore, the reduction in MZ B cell compartment was also observed by immunohistochemical analysis by containing spleen sections with anti-IgM and anti-IgD. In this case, the IgM single positive MZ which lies outside of the B cell follicles was greatly reduced with treatment in both transgenic and nontransgenic littermates (FIG. 5E). Therefore, while splenomegaly and expansion of the follicular B cell compartment are unaltered with LTBR-Ig treatment, the MZ B cell compartment is sensitive to treatment.

B-1 cells have been shown to exhibit many similarities with MZ B cells in terms of the kinds of antigen they respond to and the fact that they can persist long term when Rag or IL-7 expression is switched off in an adult mouse. Since the B-1 cell subset may express auto-reactive B cell receptors, it was of interest to determine if LTBR-Ig had an effect on this subset and whether this subset would be required for disease in the BAFF-transgenic mice. The B-1 subset was found to be expanded approximately 2-3 fold in aged BAFF-transgenic mice compared to littermate controls. Moreover, the B-1a subset appears to be slightly preferentially expanded (summarized in Table 3). Treatment with LTBR-Ig was found to have no effect on the hyper-expansion of CD23-CD5+B-1a cells analyzed from the peritoneal lavage of these mice. Therefore, improvement of GN in BAFF-transgenic mice with LTBR-Ig treatment cannot be explained by a decrease in B-1a cells.

Other B cell effector compartments were also evaluated in treated BAFF-transgenic mice (summarized in Table 3). Following an immune response, plasma cells home to the bone marrow (BM), but can also be detected in the spleen and lymph nodes. Plasma cell counts were evaluated by FACS by staining for syndecan-1. In BAFF-transgenic animals, plasma cells were increased approximately 6-fold in the spleen compared to nontransgenic littermates. LTBR-Ig treatment did not decrease the number of splenic plasma cells—in fact they were slightly increased in numbers. This is likely due to the fact that LTBR-Ig treatment often induces mild splenomegaly which in turn results in greater numbers of these cells. In the mesenteric lymph nodes (MLN) of BAFF-transgenic mice, a very large increase (35-fold) in IgA-secreting plasma cells was noted. These were reduced approximately 2-fold with LTBR-Ig treatment. In the BM, Ig secreting cells were evaluated by ELISPOT™ analysis. BAFF-transgenic mice exhibited 46-fold increase in IgG-secreting cells and a 5-fold increase in IgA-secreting cells compared to non-transgenic littermates. However, numbers of IgG- and IgA-secreting cells did not decrease with LTBR-Ig treatment.

Finally, in SLE animals, plasma cells have been observed in the kidney likely due to the expression of chemokines in this inflamed organ. Accordingly, in BAFF-transgenic mice, a significant number of IgA-secreting cells, and less so IgG-secreting cells were detected in the kidneys by ELISPOT™. The IgA-secreting cells were decreased approximately 2-fold with LTBR-Ig treatment. Taken together, of all cell subsets tested, only MZ B cells are significantly modulated by LTBR-Ig treatment, however, IgA-secreting cells in the kidney and in the MLN may be under partial LT control. This also suggests a migration defect of these effector cells to inflamed tissue. Disruption of chemokine expression may be responsible for this phenomenon and could have an important therapeutic advantage in treating SLE. Indeed, CXCR3 ligands have been recently shown to be important in plasma cell migration. Consistent with a role for the LT pathway in plasma cell migration, we have found that CXCR3 ligands can be induced by LTBR stimulation in the presence of IFN-γ.

TABLE 3

| | BAFF-Transgenic | | | | WT Littermates | | | |
|---|---|---|---|---|---|---|---|---|
| | huIgG | | LTBR-Ig | | huIgG | | LTBR-Ig | |
| Spleen weight (g) | 0.224 | (0.015) | 0.226 | (0.013) | 0.117 | (0.015) | 0.147 | (0.010) |
| Splenocyte count (×10⁷) | 28.10 | (3.83) | 30.97 | (2.66) | 6.72 | (0.62) | 14.45 | (0.43) |
| Follicular B cell count | 14.39 | (4.69) | 18.11 | (5.37) | 2.86 | (1.02) | 6.76 | (3.23) |
| (×10⁷) | | | | | | | | |
| MZ B | 9.37 | (0.54) | 0.97 | (0.20) | 2.85 | (0.29) | 0.50 | (0.17) |
| (% of B220 + splenocytes) | | | | | | | | |
| % B-1a of PEC | 17.25 | (2.44) | 16.50 | (1.18) | 7.34 | (1.61) | 5.11 | (0.99) |
| Ratio B-1a/B, 1b | 1.35 | (0.18) | 1.17 | (0.09) | 0.65 | (0.05) | 0.48 | (0.01) |
| Plasma cell count, | 17.13 | (2.69) | 33.91 | (5.24) | 2.87 | (0.21) | 8.08 | (2.29) |
| spleen (×10⁶) | | | | | | | | |
| IgA + Plasma cell count, | 3.88 | (1.06) | 1.96 | (0.60) | 0.11 | (0.07) | 0.14 | (0.08) |
| MLN (×10⁶) | | | | | | | | |
| Frequency IgA secretors, | 10.00 | (3.33) | 10.09 | (3.10) | 1.75 | (1.28) | 1.4 | (0.66) |
| spleen (per 10⁴ cells) | | | | | | | | |
| Frequency IgG secretors, | 123.00 | (4.96) | 317.27 | (8.23) | 125.00 | (6.89) | 180.00 | (10.33) |
| spleen (per 10⁴ cells) | | | | | | | | |
| Frequency IgG secretors, | 26.40 | (8.09) | 36.18 | (8.51) | 1.75 | (1.19) | 4.00 | (2.20) |
| BM (per 10³ cells) | | | | | | | | |
| Frequency IgA secretors, | 13.00 | (6.29) | 13.40 | (3.14) | 2.50 | (1.67) | 0 | (0) |
| BM (per 10³ cells) | | | | | | | | |
| Frequency IgG secretors, | 0.8 | (0.01) | 8.45 | (3.01) | 5.5 | (2.69) | 1.25 | (0.55) |
| kidney (per 10⁴ cells) | | | | | | | | |
| Frequency IgA secretors, | 36.40 | (15.57) | 18.27 | (5.89) | 0 | (0) | 0 | (0) |
| kidney (per 10⁴ cells) | | | | | | | | |

### Example 6

### Evaluation of the Role of GCs In Diseased BAFF-Transgenic Mice

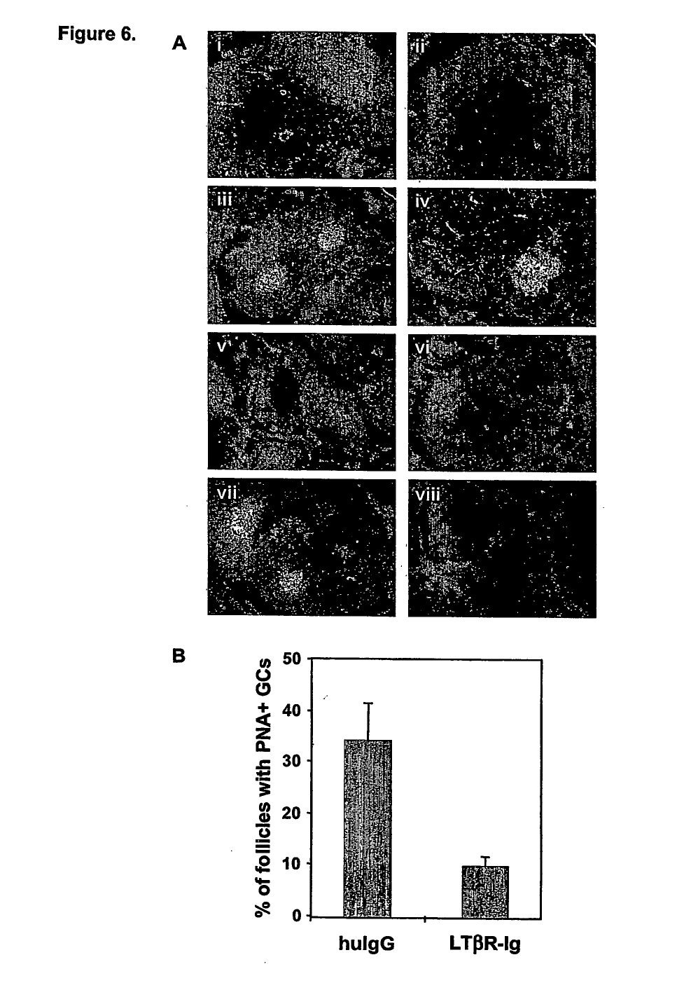

Follicular dendritic cells (FDCs) are tightly regulated by the LT pathway in both mice and in primates. Because FDCs are located within GCs and have the capacity to retain antigen, this cell type may play a role in selection of high affinity B cells during an immune response. Examination of the affinity maturation of humoral responses in LT-deficient and LTBR-Ig treated animals has yielded different conclusions that are likely dependent on the dose and route of administration of the antigen. In any case, elimination of FDC networks as a consequence of LTBR-Ig treatment may be one explanation for the improved Glumerulonephritis (GN) observed with treatment Mice were treated as described in Example 1. After 5 weeks of treatment, spleens from BAFF-transgenic mice were evaluated for PNA staining in order to enumerate GCs. BAFF-transgenic mice were observed to have a significant number of GCs, although they had never been immunized (FIG. 6A (iii)). LTBR-Ig treatment partially reduced the number of PNA-positive GCs in BAFF-transgenic spleens (P<0.006) (FIG. 6B). However, when spleens were stained with the FDC-specific marker M-1, the reduction in FDC networks with LTBR-Ig treatment was complete. Hence, although there is not a complete reduction in PNA-positive GCs, FDC networks are eliminated with LTBR-Ig treatment.

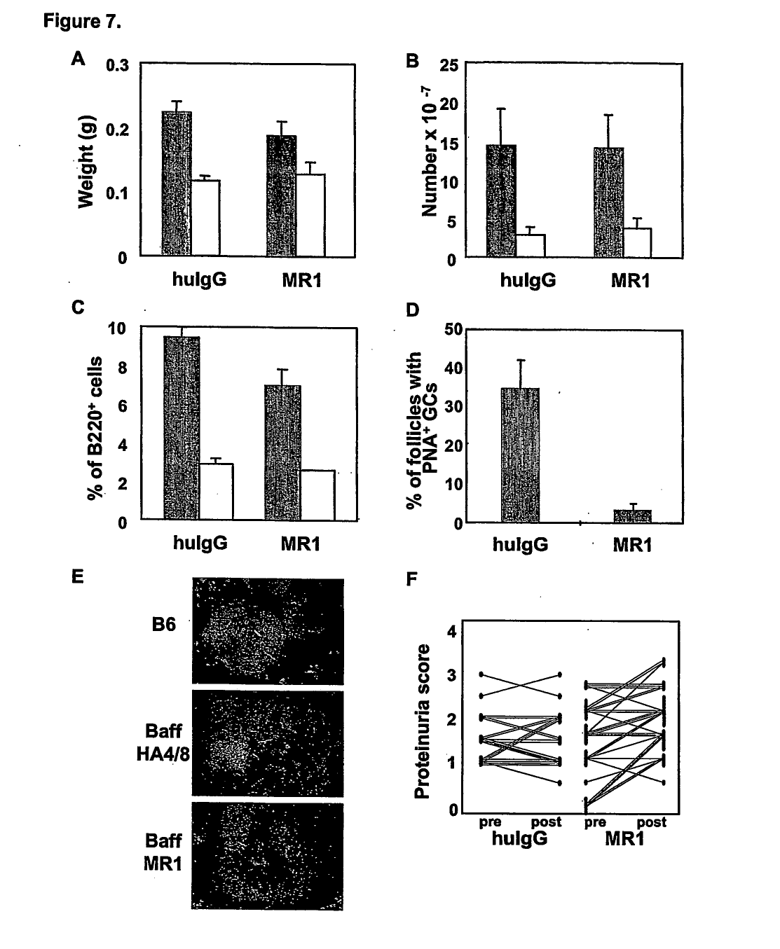

In order to determine whether this partial reduction in PNA-positive GCs was relevant to the disease reduction observed in BAFF-transgenic mice, the effect of blocking the CD40 pathway was evaluated. CD40-deficient animals are incapable of forming PNA-positive GCs, and T-dependent immune responses are completely impaired in these animals. Moreover, treatment of adult mice with an anti-CD40 ligand antibody (MR1) has a similar effect on GCs and T-dependent immune responses. Similar to LTBR-Ig treated animals, MR1 treatment had no effect on splenomegaly (FIG. 7A) nor on the increased follicular B cell compartment (FIG. 7B). However, MR1 also did not have any effect on the expanded MZ B cell compartment of BAFF-transgenic mice (FIG. 7C). MR1 treated BAFF-transgenic mice had no observable PNA-positive GCs in their spleens (P<0.00l compared to control treated BAFF-transgenics) (FIGS. 7D and 7E). In spite of this effect on the GC, MR1 treatment did not improve kidney function in diseased BAFF-transgenic mice as measured by proteinuria (FIG. 7F), nor was kidney pathology significantly improved. Taken together, in spite of its well studied effects on the GC reaction and T-dependent responses, inhibition of the CD40 pathway has no impact on disease in BAFF-transgenic mice.

The specification is most thoroughly understood in light of the teachings of the references cited within the specification which are hereby incorporated by reference. The embodiments within the specification provide an illustration of embodiments of the invention and should not be construed to limit the scope of the invention. The skilled artisan readily recognizes that many other embodiments are encompassed by the invention. All publications and patents cited in this disclosure are incorporated by reference in their entirety. The citation of any references herein is as not an admission that such references are prior art to the present invention.

1-2. (canceled)

3. A method of treating IgA nephropathy in a mammal, the method comprising administering to the mammal a therapeutically effective amount of an inhibitor of the lymphotoxin pathway selected from (a) a soluble lymphotoxin-beta receptor (LTBR), (b) an anti-LTBR antibody, and (c) an anti-LT antibody.

4. The method of claim 3, wherein the mammal is a human.

5. The method of claim 3, wherein the inhibitor is a monospecific anti-LTBR antibody or anti-LT antibody or antigen-binding domain thereof.

6. The method of claim 3, wherein the inhibitor is a soluble LTBR polypeptide.

7. The method of claim 6, wherein the polypeptide comprises at least 180 contiguous amino acids of SEQ ID NO: 1,.

8-9. (canceled)

10. The method of claim 6, 7, 26, 27, 28, 29 or 30, wherein the soluble LTBR polypeptide further comprises an Fc domain of an IgG1 or IgG4.

11. The method of claim 3, wherein the inhibitor comprises a soluble LTBR fused to one or more heterologous protein domain.

12-13. (canceled)

14. The method of claim 6, wherein the soluble LTBR polypeptide is a human LTBR polypeptide.

15. The method of claim 11, wherein the heterologous protein domain comprises a human immunoglobulin Fc domain.

16-23. (canceled)

24. A method of evaluating the efficacy of a compound for treatment of IgA nephropathy, comprising: administering the compound to a BAFF-transgenic non-human animal; determining the test level of IgA deposits in a kidney of the animal after administration; and comparing the level with a threshold level, wherein a test level lower than the threshold level indicates that the compound is efficacious.

25. The method of claim 24, wherein the animal is a rodent.

26. The method of claim 6, wherein the soluble LTBR polypeptide comprises a sequence consisting essentially of SEQ ID NO: 1.

27. The method of claim 6, wherein the soluble LTBR polypeptide comprises a sequence at least 95% identical to SEQ ID NO: 1, and wherein the polypeptide specifically binds LT and inhibits the LT pathway.

28. The method of claim 27, wherein the soluble LTBR polypeptide comprises a sequence at least 97% identical to SEQ ID NO: 1.

29. The method of claim 27, wherein the soluble LTBR polypeptide comprises a sequence at least 98% identical to SEQ ID NO: 1.

30. The method of claim 27, wherein the soluble LTBR polypeptide comprises a sequence at least 99% identical to SEQ ID NO: 1.

31. The method of claim 5, wherein the antibody is a humanized antibody.

32. The method of claim 5, wherein the antibody is a single-chain antibody.

33. The method of claim 5, wherein the antibody is a chimeric antibody.

34. The method of claim 5, wherein the antibody is a CDR-grafted antibody.

35. The method of claim 1, 5 or 6, wherein the inhibitor is administered by a route selected from the group consisting of: intravenous, intraperitoneal, intramuscular, intracavity, subcutaneous, and transdermal.

36. The method of claim 1, 5 or 6, wherein the inhibitor is administered at a dosage from 1 ug/kg to 20 mg/kg.

37. The method of claim 1, 5 or 6, wherein the inhibitor is administered at a dosage from 1 ug/kg to 10 mg/kg.

38. The method of claim 1, 5 or 6, wherein the inhibitor is administered at a dosage from 100 ug/kg-1 mg/kg.

39. The method of claim 10, wherein the inhibitor is formulated for injection.


SEQUENCE LISTING (3 sequences)

SEQ ID NO: 1 - protein, 197 aa, Homo sapiens
SQPQAVPPYASENQTCRDQEKEYYEPQHRICCSRCPPGTYVSAKCSRIRDTVCATCAENSYNEHWNYLTICQLCRPCDPVMGLEEIAPCTSKRKTQCRCQPGMFCAAWALECTHCELLSDCPPGTEAELKDEVGKGNNHCVPCKAGHFQNTSSPSARCQPHTRCENQGLVEAAPGTAQSDTTCKNPLEPLPPEMSGT

SEQ ID NO: 2 - DNA, 35 bp, Homo sapiens
taagaatgcggccgcggaatggatgagtctgcaaa

SEQ ID NO: 3 - DNA, 35 bp, Homo sapience
taagaatgcggccgcgggatcacgcactccagcaa